(19) United States
(12) Patent Application Publication
(10) Pub. No.: US 2008/0108685 A1
(43) Pub. Date: May 8, 2008

(54) Novel Use for Alpha Sympathomimetics Having a 2-Imidazoline Structure

(76) Inventors: Fritz Sacher, Gau-Algesheim (DE); Marion Tschaikin, Darmstadt (DE); Stephan Koelsch, Worms (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/664,610

(22) PCT Filed: Sep. 16, 2005
(86) PCT No.: PCT/EP05/10026

§ 371 Date: Apr. 4, 2007

(30) Foreign Application Priority Data
Oct. 5, 2004 (DE) 10 2004 049 008.2

(51) Int. Cl. A61K 31/4164; A61P 31/12

(52) U.S. Cl. 514/401

(57) ABSTRACT

The invention relates to the use of α-sympathomimetics having a 2-imidazoline structure for the preparation of a medicament for the prophylaxis and/or treatment of viral diseases.

The invention relates to the use of α-sympathomimetics having a 2-imidazoline structure for the preparation of a medicament for the prophylaxis and/or treatment of viral diseases.

α-Sympathomimetics having a 2-imidazoline structure are employed for the local treatment of swellings of the nasal mucous membrane, for example in acute rhinitis and allergic rhinitis. α-Sympathomimetics stimulate α-receptors in the vessels and thus have a strong vasoconstrictory action. On local administration into the nose, a significant constriction of the vessels in the nasal mucous membrane occurs, which then results in a reduction in mucous secretion and decongestion of the mucous membranes. This improves the passage of air and eliminates the impedance to nasal breathing.

Acute rhinitis, also known as the common cold, is an acute inflammation of the nasal mucous membrane which is caused by viruses. The above-described use of α-sympathomimetics having a 2-imidazoline structure in acute rhinitis for decongestion of the mucous membrane is based on their vasoconstrictory action described above. Their use in the treatment of acute rhinitis is thus not directed against the disease acute rhinitis per se, but instead is only a palliative treatment which is directed against the (nonspecific) symptom of mucous membrane swelling which also occurs in acute rhinitis.

Surprisingly, it has been found that α-sympathomimetics having a 2-imidazoline structure have an antiviral action and can thus be employed for the causal prophylaxis and/or treatment of viral diseases. The invention therefore relates to the use of α-sympathomimetics having a 2-imidazoline structure for the preparation of a medicament for the prophylaxis and/or treatment of viral diseases.

α-Sympathomimetics having a 2-imidazoline structure which can be used in accordance with the present invention are all active ingredients having an α-sympathomimetic action which contain a terminal 2-substituted imidazoline ring, such as, for example, naphazoline, tramazoline, tetryzoline, fenoxazoline, xylometazoline or oxymetazoline. The medicament according to the invention which is intended for the prophylaxis and/or treatment of viral diseases may comprise one or more α-sympathomimetic(s) having a 2-imidazoline structure. Particular preference is given to the use of oxymetazoline and/or xylometazoline, very particularly preferably oxymetazoline.

For the purposes of the invention, viral diseases are all diseases which are caused by viruses, such as, for example, herpes (herpes simplex virus), pneumonia (Sendai virus), inclusion disease (cytomegalovirus), influenza (influenza and parainfluenza viruses), infectious mononucleosis/Pfeiffer's disease (Epstein-Barr virus), AIDS (human immunodeficiency virus/HIV), enteritis (rotaviruses, Norwalk virus, adenoviruses, enteroviruses), hepatitis (hepatitis viruses), meningitis (Coxsackie viruses, ECHO viruses, inter alia) encephalitis (arboviruses, ECHO viruses, inter alia), follicular conjunctivitis (adenoviruses, herpes viruses, inter alia), respiratory tract infections (rhinoviruses, parainfluenza, respiratory syncytial virus, adenoviruses, inter alia), such as acute rhinitis, medial otitis, sinusitis, tonsillitis, pharyngitis, laryngitis, bronchitis. Viral diseases for the prophylaxis and/or treatment of which α-sympathomimetics having a 2-imidazoline structure are preferably employed are acute rhinitis, influenza, parainfluenza, conjunctivitis, medial otitis and sinusitis.

The medicament comprising one or more α-sympathomimetics can be administered preventatively, i.e. prophylactically. In this case, the viruses causing the disease are combated immediately after infection, so that the disease does not occur at all. The medicament comprising one or more α-sympathomimetic(s) can likewise also be employed after onset of the disease, i.e. for combating the viruses when they have already resulted in the disease, and thus for the treatment of the viral disease.

Depending on the use, the medicament comprising one or more α-sympathomimetic(s) can be administered systemically or topically. Systemic administration is taken to mean all administration methods which result in the active ingredient(s) being absorbed by the body after administration and distributed throughout the body via the bloodstream. This is, in particular, oral and parenteral administration, but also other types of administration, such as, for example, transdermal or pulmonary administration.

Topical administration is taken to mean all administration methods by means of which the medicaments comprising the active ingredient(s) are administered directly to body surfaces or into hollow organs on or in which they are to develop their action. Particularly suitable is administration to the skin, including the skin lining body orifices, such as, for example, in the ear, or to mucous membranes, for example in the eye, in the nose, in the throat region or in the rectal region.

Topical administration is preferred. The invention therefore relates to the use of α-sympathomimetics having a 2-imidazoline structure for the preparation of a medicament for the prophylaxis and/or preparation of viral diseases, which is characterised in that the medicament is intended for topical use.

Particular preference is given to topical use of the medicament according to the invention on mucous membranes. The invention therefore also relates to the use of α-sympathomimetics having a 2-imidazoline structure for the preparation of a medicament for the prophylaxis and/or therapy of viral diseases, which is characterised in that the medicament is intended for use on mucous membranes.

According to an advantageous embodiment of the invention, the medicament comprising one or more α-sympathomimetic(s) having a 2-imidazoline structure is employed against viral diseases in the nose/throat region, the respiratory tract, the ear, the skin and/or the eye. The invention therefore furthermore relates to the use of α-sympathomimetics having a 2-imidazoline structure for the preparation of a medicament for the prophylaxis and/or preparation of viral diseases, which is characterised in that the latter is a disease in the nose/throat region, the respiratory tract, the ear, the skin and/or the eye. The medicament is preferably employed for the prophylaxis and/or treatment of viral diseases in the nose/throat region, the ear and/or the eye. Particular preference is given to the use of the medicament for the prophylaxis and/or treatment of acute rhinitis, influenza, parainfluenza, conjunctivitis, medial otitis, sinusitis. The use of the medicament for the prophylaxis and/or treatment of acute rhinitis and/or influenza is very particularly preferred.

It goes without saying that the form of the medicament must in each case be matched to the administration method. Suitable oral administration forms can be, for example, tablets, capsules, dragees, granules or liquids, parenterals, for example solutions, emulsions or suspensions, topical administration forms, for example gels, ointments, lotions, creams, solutions, emulsions, suspensions, powders, suppositories or aerosols. The administration forms which are suitable for the particular application, the composition and preparation thereof are well known to the person skilled in the art and do not require further explanation here. Reference is made to the relevant standard works, for example H. Sucker, P. Fuchs, P. Speiser, “Pharmazeutische Technologie” [Pharmaceutical Technology], Stuttgart 1978 or K. H. Bauer, K. H. Frömming, C. Führer, “Pharmazeutische Technologie” [Pharmaceutical Technology], Stuttgart 1986. These are incorporated herein by way of reference and are thus part of the disclosure.

The α-sympathomimetics having a 2-imidazoline structure can be employed as the base or also as the acid-addition salts thereof with inorganic acids, for example sulfuric acid, sulfurous acid, dithionic acid, nitric acid, hydrohalic acids, such as hydrochloric acid or hydrobromic acid, phosphoric acids, such as, for example, orthophosphoric acid, or sulfamic acid, or with organic acids, such as, for example, formic acid, acetic acid, pivalic acid, malonic acid, succinic acid, pimelic acid, fumaric acid, maleic acid, lactic acid, tartaric acid, malic acid or citric acid. Particular preference is given to the use of the acid-addition salts and in turn here the hydrochlorides. This applies, in particular, if use is made of aqueous solutions, which are preferred.

According to an advantageous embodiment of the invention, use is made of a medicament which additionally comprises one or more zinc salts. Zinc salts which may be present are in principle all pharmaceutically acceptable zinc salts, preferably zinc chloride, zinc lactate, zinc sulfate, zinc citrate, zinc acetate, zinc histidinate, zinc orotate, zinc aspartate and/or zinc gluconate. The medicament particularly preferably comprises zinc gluconate.

According to a preferred embodiment of the invention, use is made of a medicament which is characterised in that the α-sympathomimetic(s) is (are) present in an aqueous solvent. Aqueous solutions are, for example, particularly suitable for topical use on mucous membranes, such as, for example, in the eye in the nose/throat region or in the rectal region, and are particularly preferably employed in the nose for the treatment of acute rhinitis.

The aqueous solutions may comprise adjuvants, such as, for example, buffers and preservatives. Suitable buffers are in principle all physiologically tolerated substances which are suitable for setting the desired pH, such as, for example, citrate salts, acetate salts, histidine salts succinate salts, malate salts, phosphate salts or lactate salts, and/or the respective free acids or bases thereof as well as mixtures of the various salts and/or acids or bases thereof. Aqueous solutions comprising α-sympathomimetics having a 2-imidazoline structure particularly preferably comprise phosphate or citrate buffer, in particular if oxymetazoline and/or xylometazoline is/are present as active ingredient. According to a particularly preferred embodiment of the invention, the medicament that comprises one or more α-sympathomimetic(s) and is intended for the prophylaxis and/or treatment of viral diseases is in the form of an aqueous solution and comprises at least one buffer, preferably phosphate or citrate buffer.

Suitable phosphate buffers are solutions of the mono- and/or disodium and potassium salts of phosphoric acid, such as disodium hydrogenphosphate or potassium dihydrogenphosphate, and mixtures of the sodium and potassium salts, such as, for example, mixtures of disodium hydrogenphosphate and potassium dihydrogenphosphate.

Suitable citrate buffers are mixtures of one or more citrate salt(s) and/or the free acid thereof (for example citric acid, citric acid monohydrate, trisodium citrate dihydrate, tripotassium citrate monohydrate).

The pH of the aqueous solution is in the range from 4.0 to 8.0, preference is given to a pH of 5.5 to 7.0. If the aqueous solution comprises phosphate buffer, this is advantageously present in a concentration of 5 to 180 mmol/l, preferably 140 to 145 mmol/l. If citrate buffer is present, this is advantageously present in a concentration of 1 to 50 mmol/l, preferably 5 to 20 mmol/l.

Suitable preservatives are, for example, phenol, m-cresol, methyl- or propylparaben, chlorobutanol, thiomersal or benzalkonium chloride, of which benzalkonium chloride is preferred, in particular if an aqueous solution is intended for topical use on mucous membranes and here in particular for use in the nose. According to a further preferred embodiment of the invention, the medicament that comprises one or more α-sympathomimetic(s) and is intended for the prophylaxis and/or treatment of viral diseases is in the form of an aqueous solution and comprises at least one preservative, preferably benzalkonium chloride.

If the solution is not already isotonic due to the osmotic properties of the active ingredient and the adjuvants furthermore present, an isotonic agent, preferably a physiologically tolerated salt, such as, for example, sodium chloride or potassium chloride, or a physiologically tolerated polyol, such as, for example, glucose or glycerol, may advantageously furthermore be present in an amount necessary for isotonicisation. The isotonic agent is particularly preferably glycerol.

Depending on the α-sympathomimetic present, the administration form and the particular viral disease, the medicament may be intended for administration one or more times weekly to one or more times daily. According to an embodiment of the invention, the medicament is intended for administration twice, three times or more than three times daily.

The examples explain the invention without being restricted thereto.

### EXAMPLE

### Antiviral Activity

The antiviral activity of the α-sympathomimetics having a 2-imidazoline structure is investigated in a series of in-vitro experiments with the example of oxymetazoline against the viral strains HRV 2 and HRV 14 (human rhinovirus) and influenza A. The investigations are carried out on HeLa cells infected with HRV 2 or HRV 14 or MDCK cells infected with influenza A. Here, the influence of oxymetazoline on virus replication/virus reproduction is demonstrated by the plaque reduction assay (see Cooper, P. D., 1955: A method for producing plaques in agar suspensions of animal cells. Virologiy 1:397-409) and on the infectiousness of viruses (determination of the residual infectiousness by virus titration). In addition, antiviral action properties were measured by the so-called high-throughput cytopathic effect inhibitory assay (CPE) (Schmidtke M., Schnittler U., Jahn B., Dahse H.-M. and Stelzner A. 2001: A rapid assay for evaluation of antiviral activity against coxsackie virus B3, influenza virus A, and herpes simplex virus type 1. J Virol Methods 95: 133-143).

### Virus Strains HRV 2 and HRV 14

In order to exclude cytotoxic effects caused by the active ingredient, firstly the active-ingredient concentration at which no influence on the HeLa cells present arises under the given in-vitro test conditions is determined by addition of a dilution series of aqueous active-ingredient solutions (see Mosmann, T., 1983: Rapid Colorimetric Assay for Cellular Growth and Survival: Application to Proliferation and Cytotoxicity Assays. J. Immunol. Meth. 65:55-63). The active-ingredient concentration at which no effects caused by the test substances which reduce the metabolism of the HeLa cells arise is between 0.005 and 0.003125% (W/V).

The infection of the HeLa cells with HRV 14 is carried out using an average infection dose (M.O.I., multiplicity of infection) of 0.0004. For infection of the HeLa cells with HRV 2, a virus dose was selected which resulted in complete CPE in the untreated virus controls 72 hours after infection. The test substance is added in dilutions (1:2, 1:4, 1:8, 1:16, 1:32) of the determined non-cytotoxic substance concentration of 0.003125% (W/V) to the infected cells. The antiviral activity is measured with the aid of the plaque reduction test and/or the high-throughput cytopathic effect inhibitory assay (CPE). The residual infectiousness (TCID₅₀/ml) is determined by means of virus titration. The HRV 14 results are shown in Tables 1 and 2.

### HRV 14

TABLE 1

| | | Residual | |
|---|---|---|---|
| Dilution | Plaque reduction | infectiousness | Rel. total |
| (1/X) | rel. inhibition [%] | rel. inhibition [%] | inhibition |
| 2 | 44.26 | 33.33 | 38.79 |
| 4 | 32.98 | 20.83 | 26.91 |
| 8 | 25.74 | 18.75 | 22.25 |
| 16 | 17.66 | 18.75 | 18.20 |
| 32 | 14.26 | 12.50 | 13.38 |

The results show by way of example for oxymetazoline the antiviral action of the α-sympathomimetics having a 2-imidazoline structure (here against virus strain HRV 14).

TABLE 2

| Dilution | |
|---|---|
| (1/X) | CPE inhibition [%] |
| 2 | 50.5 |
| 4 | 53.6 |
| 8 | 37.5 |
| 16 | 5.9 |

### Results of a Second Series of Experiments with Virus Strain HRV 14 (Measurement by Means of CPE Assay)

The results for HRV 2 are shown in Table 3.

### HRV 2

TABLE 3

| Dilution | |
|---|---|
| (1/X) | CPE inhibition [%] |
| 2 | 40.5 |
| 4 | 23.2 |

The results show by way of example for oxymetazoline the antiviral action of the α-sympathomimetics having a 2-imidazoline structure (here against virus strain HRV 2).

### Influenza A Virus

Analogously to the process described for the two rhinoviruses (HRV 2, HRV 14), firstly the active-ingredient concentration at which no influence on the MDCK cells used arises under the given in-vitro test conditions is determined by addition of a dilution series of aqueous active-ingredient solutions. The active-ingredient concentration at which no effects caused by the test substances which reduce the metabolism of the MDCK cells arise is 0.005% (W/V). The test substance is added in dilutions (1:2, 1:4, 1:8, 1:16, 1:32) of the determined non-cytotoxic substance concentration of 0.005% (W/V) to the infected cells. The antiviral activity is quantified with the aid of the high-throughput cytopathic effect inhibitory assay (CPE). The results are shown in Table 4.

TABLE 4

| Dilution | |
|---|---|
| (1/X) | CPE inhibition [%] |
| 2 | 52.8 |
| 4 | 35.5 |
| 8 | 23.1 |
| 16 | 9.1 |

The results show by way of example for oxymetazoline the antiviral action of the α-sympathomimetics against the influenza A virus strain (CPE assay).

1. Use of α-sympathomimetics having a 2-imidazoline structure for the preparation of a medicament for the prophylaxis and/or treatment of viral diseases.

2. Use according to claim 1, characterised in that the α-sympathomimetic(s) having a 2-imidazoline structure that is used is oxymetazoline and/or xylometazoline.

3. Use according to claim 1, characterised in that the medicament is intended for topical use.

4. Use according to claim 3, characterised in that the medicament is intended for use on mucous membranes.

5. Use according to claim 1, characterised in that the viral disease is a disease in the nose/throat region, the respiratory tract, the ear, the skin and/or the eye.

6. Use according to claim 5, characterised in that the viral disease is a disease in the nose/throat area, the ear, and/or the eye.

7. Use according to claim 6, characterised in that the viral disease is acute rhinitis, influenza, parainfluenza, conjunctivitis, medial otitis, sinusitis.

8. Use according to claim 1, characterised in that the α-sympathomimetic(s) is (are) present in an aqueous solvent.

9. Use according to claim 8, characterised in that the aqueous solution comprises at least one buffer, preferably phosphate or citrate buffer.

10. Use according to claim 8, characterised in that the aqueous solution comprises at least one preservative, preferably benzalkonium chloride.
